(19) United States
(12) Patent Application Publication
(10) Pub. No.: US 2002/0110845 A1
(43) Pub. Date: Aug. 15, 2002

(54) Method for the prediction of starch digestion

(75) Inventors: Henri Hansson, Helsingborg (SE); Christer Spegel, Lund (SE)

Correspondence Address: Patricia Coleman James, McCutchen, Doyle, Brown & Enersen, LLP, 18th Floor, Three Embarcadero Center, San Francisco, CA, 94111, US

(73) Assignee: Metcon Medicin AB

(21) Appl. No.: 10/002,527

(22) Filed: Oct. 25, 2001

Related U.S. Application Data
(60) Provisional application No. 60/243,070, filed on Oct. 25, 2000.

(30) Foreign Application Priority Data
Oct. 25, 2000 (SE) 0003876-0

(51) Int. Cl. C12Q 1/40

(52) U.S. Cl. 435/22

(57) ABSTRACT

The degradation resistance properties of native starch are studied in vitro using a photometrical method. The reducing sugars, formed by the enzymatic degradation of the starch molecules, form complexes with 3,5-dinitro salicylatc, the concentration of which can be spectrophotometrically determined. The method has exhibited good accuracy and high reproducibility. The obtained results can be used to predict the enzymatic degradation behavior in vivo, e.g. for predicting the degradation profile of various native starch formulations.

### CROSS REFERENCE TO RELATED APPLICATIONS

This application claims the benefit of U.S. Provisional Application No. 60/243,070, filed Oct. 25, 2000 and Swedish Application No. SE 0003876-0 filed Oct. 25, 2000.

The present invention concerns an analytical method for determining the resistance to degradation of native starch and for accurately predicting—relying on in vitro tests—the speed of digestion or digestive profile of native starch in the gastrointestinal tract of mammals.

### BACKGROUND OF THE INVENTION

Starch is the main storage polysaccharide in plants, an important source of carbohydrates and an ingredient in food. More than half of the carbohydrates ingested by humans is starch. Starch also has many technical uses, accounting for large volumes of the starch produced. Examples include the use as a sizing agent in paper industry, a raw material and/or additive in the production of plastics, textiles etc. and as a carrier and bulk agent in pharmaceutical industry. When heat treated, e.g. boiled, the starch granules disintegrate and the starch is solubilised and gelatinised. Cornstarch consists of granules sized 2-32 μm, mainly comprising two components, amylose and amylopectin. Amylose has a linear structure while arnylopectin is branched. Both arnylose and amylopectin consist of cc-(1,4)-linked glucose residues while amylopectin also has α-(1,6)-linked glucose residues.

The starch granules are insoluble in cold water and swell in warm. The swelling is reversible until the temperature reaches about 55 to 65° C. At this temperature the starch granules gelatinise and loose their crystalline structure. Gelatinised starch is then easily degraded by digestive enzymes present in the gastrointestinal tract, mainly by the action of a-amylase. In humans, a-amylase is present in the saliva and in the small intestine.

The digestibility of starch, both in vivo and in vitro, depends on the source of starch as well as its pre-treatment (e.g. native, fine/coarse, gelatinised or chemically modified). In the present description, claims and examples, the term “native starch” is used to define starch, that has not been subjected to heat-treatment or chemical treatment. The terra “native starch” thus comprises both the vegetable and/or plant seeds, kernels or grains; as well as mechanically treated fractions, such as the milled and sieved product, granules and flour.

Native starch is however extremely slowly degraded in the human gastrointestinal tract. T'nis is due to the three dimensional shape of the starch granules, making it difficult for the enzymes to access the carbon chains of the starch molecules.

### PRIOR ART

Peter Bernfield (Amylases, α and β, in Methods In Enzymology, Vol 1, page 149-158, Academic Press, New York, 1955) describes an amylase assay using 3,5-dinitrosalisylic acid as reagent, used in a photometrical determination of maltose in solution.

A method for analysing starch degradation has been disclosed by Robert L. Bruner (Determination of Reducing Value, in Methods in Carbohydrate Chemistry, 1964, pages 67-71) and has more or less remained the standard method since that time. The degradation of boiled starch by the action of α-amylase is measured as the concentration of reduced sugar in a starch suspension after the addition of an enzyme. Samples are taken at regular intervals and the reducing sugars, e.g. glucose and maltose, are reacted with a reagent; and the absorbency determined photometrically, preferably spectrophotometrically. Enzymatic degradation is then plotted as sugar concentration as a function of incubation time. This method however has many drawbacks, e.g—poor repeatability and less than satisfactory accuracy.

Stephen G. Ring et al. (Resistant Starch: Its Chemical Form In Foodstuffs and Effect on Digestability in vitro, in Food Chemistry, 28 (1988) 97-109) have studied the resistance to hydrolysis in vitro of raw and gelatinised starch from peas; maize, wheat and potatoes. Jane G. Muir and Kerin O'Dea (Validation of an in vitro assay for predicting the amount of starch that escapes digestion in the small intestine of humans, in American Journal of Clinical Nutrition, 1993; 57:540-6) have studied the digestion of gelatinised starch in eight subjects with ileostomies by determining the undigested amount remaining in ileostomy effluent.

The above prior art documents fail to put forward a fully satisfactory, accurate and useful method for determining the resistance to degradation of native starch without simultaneously (deliberately or not) denaturating the sample, and for accurately predicting—relying on in vitro tests—the speed of digestion or the digestive profile of native starch in the gastrointestinal tract of mammals.

### SUMMARY OF THE INVENTION

The present inventors have surprisingly found that the degradation resistance of native starch is more accurately measured using an analytical method as disclosed in the attached claims—The inventive method is used to compare different types of starch, different fractions of starch or different starch formulations, and is advantageously used to predict the degradation resistance properties in vivo, such as the digestion profile for different starch formulations.

### SHORT DESCRIPTION OF THE DRAWINGS

present invention will be disclosed in further detail in the following description and examples; and in the attached drawings, in which:

FIG. 1 shows the different release profile for filtered samples (solid line), compared to unfiltered samples (broken line);

FIG. 2 shows the release profile for untreated cornstarch with 0.01 M NaCl (solid line) and without NaCL (broken line) in the reaction buffer;

FIG. 3 shows the release profiles of different starch formulations, compared to untreated, native cornstarch, where the values for cornstarch granulated with ethyl cellulose (10 %) are marked with the symbol (◯), cornstarch granulated with ethyl cellulose (20%) with (Δ), cornstarch granulated with guar gum (20%) being marked with (+) and the values for untreated cornstarch being marked (×); and

FIG. 4 shows the correlation between the in vitro and in vivo behaviour of the formulations of FIG. 3, the symbols being the same,

### DESCRIPTION

The present inventors have made available a novel method for the analysis of the degradation resistance properties or digestion behaviour of native starch, wherein a predetermined amount of native starch is suspended in a buffer, a starch degrading substance added, and a reagent; forming a coloured complex with reducing sugars is added to a sample taken from the above mixture, followed by an evaluation or determination of the colour formed.

Preferably an enzyme, most preferably α-amylase, is used as the starch degrading substance.

Preferably 3,5-dinitro salicylate is used as the reagent.

Specific features of the method include the following steps:

a buffer is prepared said buffer having about neutral pH; a pH of about pH 5-pH 9, or preferably a pH of about pH 6.6 and containing a small amount chloride ions, preferably about 0.01 M chloride ions;

the test solution is incubated at a temperature below the gclatinisation temperature of the starch;

a sample is taken from the test solution and filtered before mixing with a reagent;

the absorbency is evaluated, preferably determined spectrophotometrically, and in particular measured by scanning the wavelength interval of 450 to 500 nun and the absorbency determined at the maximum value occurring within this interval.

This method is preferably used to compare different fractions of native starch with respect to their ability to resist enzymatic degradation. The method can also be used to compare different starch formulations with respect to their ability to resist enzymatic degradation. Importantly, the inventive method simulates the natural digestion of starch in the gastrointestinal tract and the values obtained can be used to reliably predict the enzymatic degradation of starch in vivo. According to the present invention, the reagent solution is prepared by dissolving 3,5-dinitro salicylate in aqueous NaOH. The reagent solution is stored in a dark place and filtered before use. This has the advantage of removing precipitate, which easily foams in the reagent solution. According to an embodiment of the invention, the reagent solution is filtered at the time of addition of the reagent to the vessels, e.g. test tubes. According to an embodiment of the invention, the reagent solution if filtered through a 0.45 μm filter. The alkaline reagent solution has the additional advantage of terminating the enzymatic activity by denaturation of the enzyme.

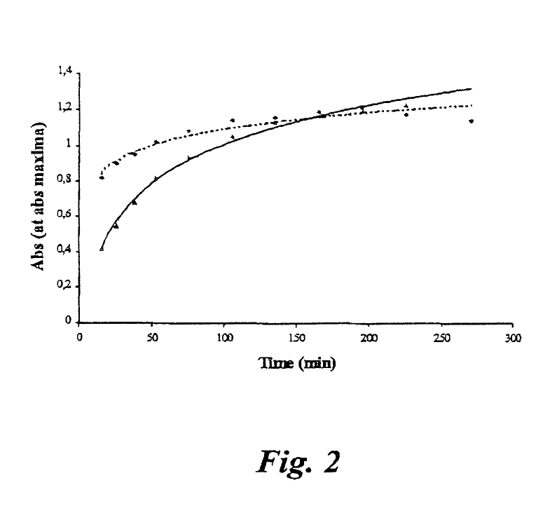

Further according to the present invention, the buffer solution is prepared by dissolving KH₂PO₄ and NaOH in water, adjusting the pH to about neutral, a pH of about pH 5 to about pH 9, or preferably a pH of about pH 6.6. NaCl is then added until the concentration of chloride ions is 0.01 M. The chloride ions have surprisingly been found to be very beneficial for the analysis, as they activate the enzyme and lead to a steeper release profile. This is an important advantage, as the time necessary for performing the analysis can be reduced. For the effect of the addition of NaCl, see FIG. 2.

Normally, enzyme is added in excess, but the present inventors have shown that the amount of enzyme added is an important factor as the enzyme has an adsorption of its own, possibly interfering with the photometric measurement. According to the invention, the enzyme is added in an amount approximately corresponding to 4 IU/mg sugar.

The enzyme chosen is an cc-amylase of fungal, bacterial or animal origin. Commercially there are four types of a-amylases available: pancreatic (porcine), fungal, bacterial (normal temperature) and bacterial (high temperature). Providers of suitable enzymes include the National Enzyme Company, USA (fungal α-amylases produced by Aspergillus oryzae), Deerland Corporation, USA (bacterial α-amylases produced by Bacillus licheniformis). The sample, a pre-determined amount of the starch to be analysed, is suspended in buffer, whereupon the vessels containing the sample-buffer suspension are placed in the incubation bath. According to the present invention, the samples are incubated at a temperature below the gelatinisation temperature of the starch; a temperature interval of 15° C.-70° C., preferably about 35° C.-70° C., and most preferably at about 37° C.—about 42° C., for example at 37° C.±0.5° C. This not only simulates the temperature in the gastrointestinal tract more accurately than the prior art methods, conducted at room temperature, it also has the additional benefit of accelerating the enzymatic action and makes the determination less time consuming. Notably, most enzymes have a temperature optimum above the temperature chosen as most preferred by the present inventors. The reduction in enzyme activity has instead been compensated for by adjusting other parameters, such as the addition of NaCl, the incubation time, filtering of the sample etc.

According to the inventive method, a sample is taken at time “zero”, i.e. before addition of the enzyme solution. A “blank” or “0 min” is taken by mixing reagent and water. Further, a sample containing all the ingredients; buffer, reagent, enzyme and starch, is taken soon after addition of the starch, at about 1 to 20 minutes after addition of the enzyme. Preferably this sample is taken 2-10 minutes, and most preferably 5 minutes after addition of the enzyme—This called the “5 min sample” later in the attached Example. Further samples are taken at predetermined intervals, e.g. at 10, 20, 30, and 45 minutes, at 1 h, 1.5 h, 2 h, 2.5 h, 3 h, 3.5 h, and 4 h after addition of the enzyme. Obviously different intervals can be chosen, depending on the purpose of the study. Similarly, the study can be continued longer than the above 4 hours, e.g. 8, 10 or 12 hours or longer.

According to an embodiment of inventive method, particularly suited for laboratory or industrial application, a “blank” or “0 min” sample is first taken by mixing reagent and buffer. The starch sample is added. After a few minutes of equilibration, the “¹0 ruin” sample is taken. The “0 min” sample will show if the starch sample contained any short sugar chains, such as glucose and maltose. Buffer and enzyme are mixed in another vessel. A blank sample is taken. The starch sample is added and time keeping is started. Further samples are taken at a pre-determined time point, e.g. at 90 minutes. Obviously different time points can be chosen, depending on the purpose of the study. Similarly, the study can be continued longer than the above 90 minutes, e.g. 8, 10 or 12 hours or longer.

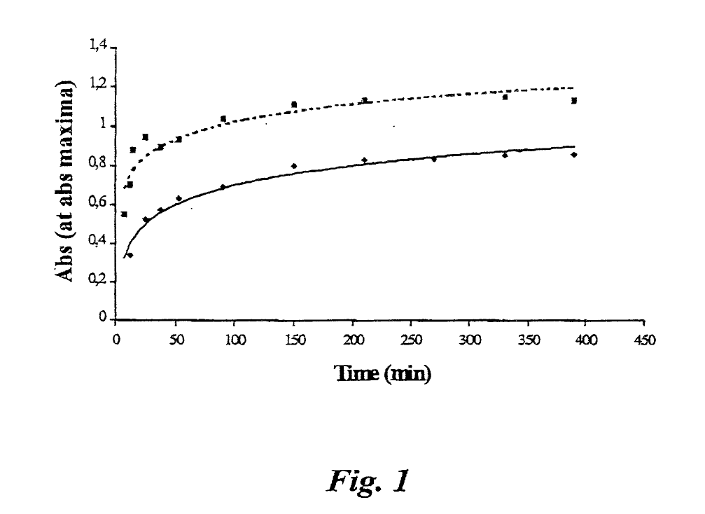

According to a preferred embodiment of the inventive method, also the sample aliquots are filtered before the evaluation or measurement of absorbency. Preferably the samples are filtered though a 0.8 p.m filter. For the effect of the filtration, see FIG. 1. It was surprisingly seen, that filtering drastically improves the accuracy of the measurement.

According to one embodiment, each absorbency measurement is conducted in the form of a scan over the wavelength interval 450-500 nm, detecting the absorption maximum. The absorbency reading is then taken at this maximum. This constitutes a significant improvement over prior art measurements, e.g. analyses using only a fixed measurement wavelength e.g. 590 nm. The inventive method involving a scan, followed by measurement at the absorption maximum has lead to an improvement in accuracy and repeatability. It should be recognised, that the photometric determination involves many sources of error and inaccuracies, as the samples contain a variety of compounds ranging from mono—and disaccharides to macroscopic fragments of starch. The scanning step introduced by the present inventors overcomes these sources of errors and offers improved reliability.

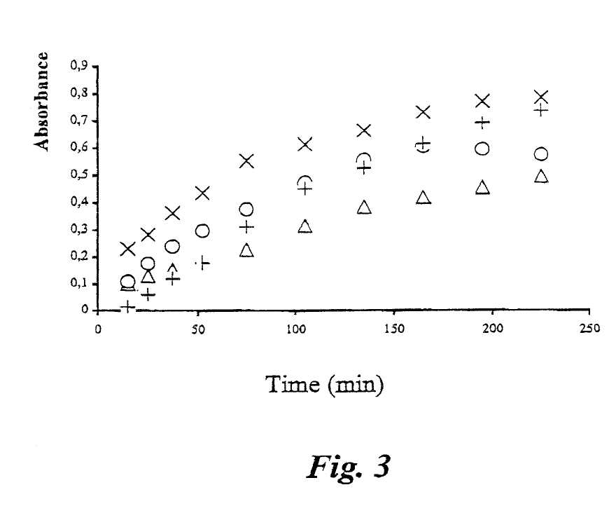

The degradation profile for native cornstarch granules is used as a standard. Depending on the purpose of the investigation, any starch fraction can be used as a standard or for comparative purposes. FIG. 3 illustrates a comparison, in that it shows the release profiles of three different starch compositions plotted in the same diagram, together with the release profile obtained for untreated cornstarch under the same conditions.

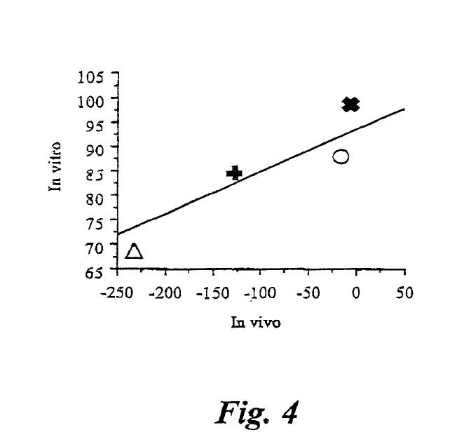

The advantages of the present invention include, but are not limited to, the easy handling and good repeatability and reliability of the analysis. Further, the inventive method provides an analytical method, well suited for practical studies of the digestibility of different types of starch, different fractions and/or qualities within the same species. Additionally, the inventive method constitutes a practical in vitro method, the results of which correlate closely with results obtained in vivo as seen in FIG. 4.

### EXAMPLES

### Example 1

### An Analysis Spanning 4 Hours

A reagent was prepared by dissolving 3,5-dinitro salicylate (2.00 g, Aldrich) in aqueous NaOH (70 ml, 1 M). Optionally, the mixture is heated in order to expedite the formation of a clear solution. Upon cooling, water is added to 100 ml. The reagent solution is stored in a dark place and filtered through a 0.45 μm filter before use, in order to remove possible precipitates. The reagent solution was added in equal amounts (2 ml) in test tubes marked “control”, “zero” “5 min” “10 min” “20 min” “30 min” “45 min” “1 h” “1.5 h” “2 h” “2.5 h” 3 h”, “3.5 h”, and “4 h”. The test tubes were placed in an ice bath awaiting the analysis.

A buffer solution (pH 6.6) was made by mixing KH₂PO₄ (250.0 ml, 0.20 M, Sigma) and NaOH (89.0 ml, 0.20 M) and adding water to a total volume of, 1000 ml. NaCl (0.58 g, Riedel-de Haën) was then added to produce a chloride concentration of 0.01 M.

According to an embodiment of the present invention, the enzymatic degradation properties of untreated or native starch granules of a known fraction are used for comparative purposes. Preferably native or untreated cornstarch granules of a known fraction are used. A defined amount of starch to be investigated (in this experiment 4.0 g) is measured and suspended in the above buffer and placed in the degradation bath. The degradation bath is kept at a temperature of 37° C.±0.5° C. and stirred at a speed of 50 rpm. In the present example, 4.0 g untreated cornstarch (Maizena, Bestfood Nordic AB) was used.

An amount corresponding to 15000 N α-amylase (Type VI-B from porcine pancreas, Sigma) is measured and suspended in buffer into the degradation bath. Before addition of the enzyme solution, a sample of the degradation bath is taken in order to determine the sugar concentration at “time zero”. The sample is filtered through a 0.8 um filter and an aliquot (2 ml) is pipetted to the test tube marked “zero”. The same filter can be used throughout the series. The sample is boiled momentary (5 min) and placed in an ice-bath. The enzyme solution is added to the degradation bath and the time registered. Samples are then taken at predetermined intervals, such as the times indicated on the test tubes. The control is prepared by boiling reagent (2 ml) and water (2 ml) during 5 min and placing the sample in an ice-bath. For each sample, the absorption is scanned in the interval 450-500 nm and the peak height registered for each absorption maximum. In order to determine the concentration of free sugars (FSO) in the native starch, the absorbency of the sample “zero” is measured against a background of buffer and reagent, the control (or “0 min blank”) sample. Both samples and control are diluted by adding 11.6 ml water to 400 μl sample. The reacted and diluted sample solution is not stable (the reading falling 0.1 to 0.2 absorbency units during 3 hours) so all samples are diluted slightly prior to the UV-spectrophotometric analysis.

In order to obtain a figure of the free, non-granulated starch present in the sample (and easily accessible for the enzyme), the “5 min” sample is analysed in the same manner as the control. This determination yields a measure of the amount of enzyme in the degradation bath, as the enzymes boiled with reagent absorb light at about the same wavelength as the reacted, reducing sugars. Following this, the “5 min” sample is used as background for the slower degradation of the native starch granules. This series of samples is diluted in the same manner as the control and “zero” sample, i.e. 11.6 ml water is added to 400 pl sample.

### Example 2

### An Analysis Based on Two Measurements

The procedure of Example 1 was repeated with the following modifications:

The reagent solution was added in equal amounts (2 ml) in test tubes marked “0 min”, “0 min blank”, “90 min”, and “90 min blank”. The test tubes were placed in an ice bath awaiting the analysis.

For the 0 min sample, a sample was taken from the buffer medium in the digestion bath. The sample was filtered and reacted with reagent as above. The blank sample was taken before addition of the starch sample.

Both samples and control were diluted by adding 8.7 ml water to 300˜.1 sample. The “90 min blank” sample is used as background for the slower degradation of the native starch granules. This series of samples is diluted in the same manner as the “0 min” and the “0 min blank” samples, i.e. 8.7 ml water is added to 300 μl sample.

Optionally, also the “control” and the “zero” readings can be used for determination of the background and/or to obtain a complete degradation profile.

As the reacted samples loose stability when diluted, all samples should be diluted and analysed in sequence, unless the spectrophotometer has the ability to store a background scan. Undiluted samples are however stable during several hours.

The results of the spectrophotometric readings are then plotted as the absorbency as a function of sample time. Alternatively a “sliding” average can be used. Using this later method, the closest previous and next cc-ordinate (time, absorbency) is averaged, and the new point is introduced at the new co-ordinates. This way the scattering of the results in relation to the regression line is decreased and the changes in the equation of the line becomes negligibly small.

In FIG. 1 it is shown how the regression of the release profile improves for filtered samples (solid line), compared to unfiltered samples (broken line), The samples were filtered through a 0.8 μm filter, the remaining experimental protocol being the same.

In FIG. 2 it can be clearly seen how the presence of a small amount of chloride ions, in this case 0.01 M, results in a steeper release profile (solid line) than the profile plotted for the degradation in absence of chloride (broken line). The experimental procedure was as described above.

FIG. 3 shows the release profiles for different starch formulations compared to native cornstarch (Maizena, Bestfood Nordic AB). In this figure, the values for cornstarch encapsulated in ethyl cellulose (10%) are marked with the symbol (◯), cornstarch encapsulated in ethyl cellulose (20%) with (▴), cornstarch encapsulated in guar guru (20%) being marked with (+) and the values for untreated cornstarch being marked (×).

In FIG. 4 the good correlation between the in vitro and in vivo behaviour of the above tested formulations is shown, the symbols being the same.

The in vivo behaviour was determined by measuring the blood glucose response according to the standard technique in 4 healthy, lean volunteers (age 35 to 45 years) with normal glucose tolerance. According to the “golden standard” of this technique, each substance was studied twice in each volunteer, and the mean value was calculated. The substances were tested in randomised order, at least one day apart. Moreover, the testing was performed under strictly standardised conditions. The subjects came to the laboratory in the morning, fasted for 10 hours. Physical activity was avoided right before and during the test. The test subjects were allowed to drink about 2 dl liquid, free from carbohydrates (water, tea or coffee) twice during the test; at 0 and 3 hours.

The capillary blood glucose level was determined in capillary blood samples (obtained by finger pricking) using a Glucometer DEX (Bayer Diagnostica AB) following the standard procedures for glucose measurements. At baseline, three consecutive blood glucose determinations were performed to ensure a stable baseline at time 0 hours. Thereafter the test substance (20.0 g) was ingested together with a standardised amount of water within 5 minutes. All liquids were carefully weighed and the same amounts ingested at each occasion to avoid variations in transit time through the gastrointestinal tract. The blood glucose determination was repeated at 0.5, 1.0, 1.5, 2.0, 3.0, and 4.0 hours.

By collecting data using the inventive analysis method, standard curves can be prepared, making it possible to make later evaluations and predictions based on fewer measurements.

Although the invention has been described with regard to its preferred embodiments, which constitute the best mode presently known to the inventors, it should be understood that various changes and modifications as would be obvious to one having the ordinary skill in this art may be made without departing from the scope of the invention as set forth in the claims appended hereto.

1. Method for the analysis of the degradation resistance of native starch, wherein a pre-determined amount of native starch is suspended in a buffer, a starch degrading substance added, a reagent added, said reagent forming a coloured complex in the presence of reducing sugars, the components forming a test solution, characterized in that the test solution is incubated at a temperature below the gelatinisation temperature of the starch, and the colour change evaluated, without preceding heat-treatment or chemical treatment of the starch.

2. Method according to claim 1, characterized in that
the buffer used has about neutral pH and contains about 0.01 M chloride ions,
α-amylase is added to the starch suspension,
the test solution is incubated at a temperature in the interval of about 35° C.—about 70° C.,
a sample is taken from the test solution and filtered before mixing with the reagent, and—the colour change of the test solution is determined as a function of time.

3. Method according to claim 2, characterized in that
the buffer used has a pH of about pH 6.6,
the test solution is incubated at a temperature in the interval of about 37° C.—about 42° C., and
the absorbency is measured by scanning the wavelength interval of 450 to 500 nm and the absorbency determined at the maximum value occurring within this interval.

4. Method according to claim 1, characterized in that the reagent is 3,5 dinitro salicylate.

5. Method according to claim 2, characterized in that the reagent is 3,5 dinitro salicylate.

6. Method according to claim 3, characterized in that the reagent is 3,5 dinitro salicylate.

7. Method according to claim 4, characterized in that the reagent solution is filtered before use.

8. Method according to claim 1, characterized in that the enzymatic degradation properties of untreated granules of a known fraction are used for comparative purposes.

9. Method according to claim 1, characterized in that different fractions of starch are compared with respect to their ability to resist enzymatic degradation.

10. Method according to claim 1, characterized in that different starch formulations are compared with respect to their ability to resist enzymatic degradation.

11. Method according to claim 8, characterized in that the values obtained are used to predict the enzymatic degradation behavior of starch in vivo.

12. Method according to claim 9, characterized in that different fractions of starch are compared with respect to their ability to resist enzymatic degradation.

13. Method according to claim 10, characterized in that different fractions of starch are compared with respect to their ability to resist enzymatic degradation.

14. Method according to claim 8, characterized in that the values obtained are used to predict the enzymatic degradation profile of starch in vivo.

15. Method according to claim 9, characterized in that the values obtained are used to predict the enzymatic degradation profile of starch in vivo.

16. Method according to claim 10, characterized in that the values obtained are used to predict the enzymatic degradation profile of starch in vivo.
